(19) United States
(12) Patent Application Publication
(10) Pub. No.: US 2002/0106381 A1
(43) Pub. Date: Aug. 8, 2002

(54) Methods for administering recombinant adeno-associated virus virions to humans previously exposed to adeno-associated virus

(76) Inventor: Katherine A. High, Philadelphia, PA (US)

Correspondence Address: ROBINS & PASTERNAK LLP, 545 MIDDLEFIELD ROAD, SUITE 180, MENLO PARK, CA, 94025, US

(21) Appl. No.: 09/880,702

(22) Filed: Jun. 13, 2001

Related U.S. Application Data
(60) Provisional application No. 60/211,066, filed on Jun. 13, 2000.

(51) Int. Cl. A61K 48/00; A61K 39/235

(52) U.S. Cl. 424/233.1; 424/93.21

(57) ABSTRACT

The present invention provides methods for administering recombinant adeno-associated virus (rAAV) virions to a human who has preexisting antibodies to wild-type adeno-associated virus (wtAAV) due to either a previous infection with wtAAV or to a previous administration of rAAV virions. In addition, the present invention also provides methods for treating hemophilia in a human who has preexisting antibodies to wtAAV or who has anti-rAAV antibodies, the methods involving administering rAAV virions that are rendered capable of expressing a heterologous gene that encodes for a blood coagulation factor whose expression results in a therapeutic benefit to the patient.

### CROSS REFERENCE TO RELATED APPLICATIONS

This application is related to U.S. Provisional Application Ser. No. 60/211,066 filed on Jun. 13, 2000, from which priority is claimed under 37 C.F.R. § 119(e), and which application is incorporated herein by reference in its entirety.

### GOVERNMENT SUPPORT

This invention was supported in part by grants from the U.S. Government (NIH Grant Nos. R01 HL53682, R01 HL53688, R01 HL61921, and P50 HL54500) and the U.S. Government may therefore have certain rights in the invention.

### FIELD OF THE INVENTION

The present invention relates to methods of administering recombinant adeno-associated virus (rAAV) virions to a human. More specifically, the invention relates to methods in which rAAV virions are introduced into a human who has been previously exposed to adeno-associated virus (AAV) either through infection of wild-type adeno-associated virus (wt-AAV) or through a previous administration of rAAV.

### BACKGROUND OF THE INVENTION

Scientists are continually discovering genes that are associated with human diseases such as diabetes, hemophilia, and cancer. Research efforts have also uncovered genes, such as erythropoietin, that are not associated with genetic disorders but instead code for proteins that can be used to treat numerous diseases. Despite significant progress in the effort to identify and isolate genes, however, a major obstacle facing the biopharmaceutical industry is how to safely and persistently deliver therapeutically effective quantities of gene products to patients. Consequently, several gene therapy methods are currently being developed to achieve this end.

Ideally, such gene therapy methods will permit the delivery of sustained levels of specific proteins (or other therapeutic molecules) to the patient. A nucleic acid molecule can be introduced directly into a patient (in vivo gene therapy), or into cells isolated from a patient or a donor, which are then subsequently returned to the patient (ex vivo gene therapy). The introduced nucleic acid then directs the patient's own cells or grafted cells to produce the desired therapeutic product. Gene therapy may also allow clinicians to select specific organs or cellular targets (e.g., muscle, blood cells, brain cells, etc.) for therapy.

Nucleic acids may be introduced into a patient's cells in several ways, including viral-mediated gene delivery, naked DNA delivery, and transfection methods. Viral-mediated gene delivery has been used in a majority of gene therapy trials. C. P. Hodgson (1995) Biotechnology (N.Y.) 13: 222-225. The recombinant viruses most commonly used in gene therapy trials (as well as pre-clinical research) are those based on retrovirus, adenovirus, herpes virus, pox virus, and adeno-associated virus (AAV). Alternatively, transfection methods may be used for gene delivery. Although transfection methods are generally not suitable for in vivo gene delivery, they may be utilized for ex vivo gene transfer. Such methods include chemical transfection methods, such as calcium phosphate precipitation and liposome-mediated transfection, as well as physical transfection methods such as electroporation.

### AAV—Mediated Gene Therapy

AAV, a parvovirus belonging to the genus Dependovirus, has several features not found in other viruses that make it particularly well suited for gene therapy applications. For example, AAV can infect a wide range of host cells, including non-dividing cells. Furthermore, AAV can infect cells from a variety of species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides, the AAV-4 genome 4767), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, which allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, that is, it requires co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

To produce infectious recombinant AAV (rAAV) containing a heterologous nucleic acid sequence, a suitable host cell line can be transfected with an AAV vector containing the heterologous nucleic acid sequence, but lacking the AAV helper function genes, rep and cap. The AAV-helper function genes can then be provided on a separate vector. Also, only the helper virus genes necessary for AAV production (i.e., the accessory function genes) can be provided on a vector, rather than providing a replication-competent helper virus (such as adenovirus, herpesvirus, or vaccinia). Collectively, the AAV helper function genes (i.e., rep and cap) and accessory function genes can be provided on one or more vectors. Helper and accessory function gene products can then be expressed in the host cell where they will act in trans on rAAV vectors containing the heterologous nucleic acid sequence. The rAAV vector containing the heterologous nucleic acid sequence will then be replicated and packaged as though it were a wild-type (wt) AAV genome, forming a recombinant virion. When a patient's cells are infected with the resulting rAAV virions, the heterologous nucleic acid sequence enters and is expressed in the patient's cells. Because the patient's cells lack the rep and cap genes, as well as the accessory function genes, the rAAV cannot further replicate and package their genomes. Moreover, without a source of rep and cap genes, wtAAV cannot be formed in the patient's cells.

There are six known AAV serotypes, AAV-1 through AAV-6. AAV-2 is the most prevalent serotype in human populations; one study estimated that at least 80% of the general population has been infected with wtAAV-2 (Bems and Linden (1995) Bioessays 17:237-245). AAV-3 and AAV-5 are also prevalent in human populations, with infection rates of up to 60% (Georg-Fries et al. (1984) Virology 134:64-71). AAV-1 and AAV-4 are simian isolates, although both serotypes can transduce human cells (Chiorini et al. (1997) J Virol 71:6823-6833; Chou et al. (2000) Mol Ther 2:619-623). Of the six known serotypes, AAV-2 is the best characterized. For instance, AAV-2 has been used in a broad array of in vivo transduction experiments, and has been shown to transduce many different tissue types including: mouse (Podsakoff et al. U.S. Pat. No. 5,858,351, herein incorporated by reference; High and Herzog U.S. Pat. No. 6,093,392, herein incorporated by reference) and dog muscle (High and Herzog, supra); mouse liver (Couto et al. (1999) Proc Natl Acad Sci USA 96:12725-12730; Couto et al. (1997) J Virol 73:5438-5447; Nakai et al. (1999) J Virol 73:5438-5447; and, Snyder et al. (1997) Nat Genet 16:270-276); mouse heart (Su et al. (2000) Proc Natl Acad Sci USA 97:13801-13806); rabbit lung (Flotte et al. (1993) Proc Natl Acad Sci USA 90:10613-10617); and rodent photoreceptors (Flannery et al. (1997) Proc Natl Acad Sci USA 94:6916-6921). Investigators have exploited the broad tissue tropism of AAV-2 to deliver tissue-specific transgenes. For example, AAV-2 vectors have been used to deliver the following genes: the cystic fibrosis transmembrane conductance regulator gene to rabbit lungs (Flotte et al., supra); Factor VIII gene (Burton et al. (1999) Proc Natl Acad Sci USA 96:12725-12730) and Factor IX gene (Nakai et al., supra; Snyder et al., supra; High and Herzog, supra) to mouse liver, dog, and mouse muscle (High and Herzog, supra); erythropoietin gene to mouse muscle (Podsakoff et al., supra); vascular endothelial growth factor (VEGF) gene to mouse heart (Su et al., supra); and aromatic 1-amino acid decarboxylase gene to monkey neurons (Bankiewicz et al., supra). Expression of certain rAAV-delivered transgenes has been shown to have therapeutic effect in laboratory animals; for example, expression of Factor IX was reported to have restored phenotypic normalcy in dog models of hemophilia B (High and Herzog, supra). Moreover, expression of rAAV-delivered VEGF to mouse myocardium resulted in neovascular formation (Su et al. supra), and expression of rAAV-delivered AADC to the brains of parkinsonian monkeys resulted in the restoration of dopaminergic function (Bankiewicz et al., supra).

### AAV Readministration

One apparent shortcoming of AAV in gene therapy is that readministration of AAV in experimental animals results in greatly reduced transduction efficiency. For example, Moskalenko et al. (J Virol (2000) 74:176101766) reported that five naïve mice all expressed hFIX for the 62-day evaluation period after receiving AAV-hFIX, while none of the twenty mice that were previously exposed to AAV-lacZ expressed Factor IX upon rAAV-hFIX administration (including those mice that were transiently immunosuppressed). Halbert et al. ((1998) J Virol 72:9795-9805) and Wilson et al. ((1999) J Virol 73:3994-4003) reported similar results. Others have demonstrated successful transduction upon readministration of rAAV-2 virions into experimental animals, but only after these animals were immune suppressed at the time of primary infection. (Wilson et al. ((2000) J Virol 74:2420-2425; Halbert et al. (1998) J Virol 72:9795-9805; Manning et al., (1998) Hum Gene Ther 9:477-85).

Unfortunately, immune suppression at the time of primary AAV infection is not possible for many humans because AAV-2 neutralizing antibodies are prevalent in human populations (Parks et al. (1970) J Virol 2:716-722). At least 80% of the general population has been infected with wtAAV-2 (Berns and Linden, supra). Moreover, readministration of rAAV virions may be necessary to achieve maximum therapeutic efficacy. In view of these observations, it has not been known whether rAAV can be delivered to more than the subset of unexposed human patients. Further, AAV-2 neutralizing antibodies can cross-react with other AAV serotypes (Erles et al. (1999) J Med Virol 59:406-411). Therefore, it has also not been known whether other rAAV serotypes could be administered to circumvent the effect of anti-AAV-2 antibodies.

Although AAV has several desirable characteristics for delivering genes to patients, there has remained significant doubt regarding whether rAAV virions can be successfully delivered to humans with preexisting anti-wtAAV antibodies, or into humans previously treated with rAAV. Existing pre-clinical animal data have shown that pre-existing and/or neutralizing antibodies to AAV inhibit rAAV transduction. Therefore, given the widespread occurrence of anti-wtAAV antibodies in the general population, and given the potential need to readminister rAAV, it would be a significant advancement in the art to provide methods for the successful delivery of rAAV to humans previously exposed to either wt-AAV or rAAV. Such methods are disclosed herein.

### SUMMARY OF THE INVENTION

One object of the present invention is to provide methods for the delivery of rAAV virions to humans having preexisting antibodies to any of the several wtAAV serotypes. It is also an object of the present invention to provide methods for the readministration of such rAAV virions to humans previously administered rAAV virions.

In one embodiment of the invention, rAAV-2 virions are delivered to humans with preexisting antibodies to any of the wtAAV or rAAV serotypes. In another embodiment, other recombinant AAV serotypes, such as rAAV-6, are delivered to humans with preexisting antibodies to any of the wtAAV or rAAV serotypes.

Recombinant AAV virions can be delivered via one or more of several routes of administration. In one embodiment, rAAV virions are delivered into muscle cells or tissue, preferably to one or more slow-twitch fibers of the muscle. Alternatively, rAAV virions are delivered directly into the bloodstream, preferably through a peripheral artery or vein. In another embodiment, rAAV virions are delivered into the body via a ductal system, including, without limitation, through the bile duct system or through the ducts of the submandibular gland or the liver.

Several different modes of administration are contemplated by the present invention. For instance, the rAAV virions can be delivered by intramuscular or subcutaneous injection. Alternatively, the rAAV virions can be delivered intravenously or intra-arterially via a catheter or simple injection.

The rAAV virions delivered according to the present methods can contain a heterologous nucleic acid sequence that codes for therapeutic anti-sense RNA molecules, ribozymes, or genes encoding particular proteins. In a preferred embodiment, the nucleic acid sequence comprises a gene encoding a blood coagulation factor, particularly Factor IX.

The invention also specifically provides methods for treating hemophilia in a human having preexisting antibodies to any of the several wtAAV or rAAV serotypes. The methods include delivering at least one rAAV virion carrying a gene encoding a blood coagulation factor, whose expression level is sufficient to provide a therapeutic effect. In a preferred embodiment, the blood coagulation factor is Factor IX, and the therapeutic effect is a reduction in the usage of recombinant Factor IX or Factor IX concentrate and/or a reduction in activated partial thromboplastin time (aPTT). dr

### BRIEF DESCRIPTION OF THE DRAWINGS

FIGS. 1a-1c show histochemical analyses of muscle tissue from a biopsy. Immunoperoxidase staining of F.IX is shown for cross-sections of muscle tissue of a negative control (a) and for a vector-injected patient (b). The dark brown staining for F.IX is seen in the extracellular matrix surrounding the muscle fibers. Original magnification: 200 X. (c) Hematoxylin and eosin (H&E) stained cross-section of muscle tissue from vector-injected patient. Original magnification: 100 X. Muscle biopsies were performed 2-3 months after vector administration.

FIGS. 2a and 2b depict immune responses to AAV-CMV-hF.IX. (a) Western blot analysis of anti-human F.IX in serum samples of hemophilia B patients. Plasma-derived hF.IX was transferred to a membrane, which was incubated with serum samples from patients. Lanes 1-2: Positive control (+) (patient with inhibitory anti-hF.IX antibodies) diluted 1:2000; Lane 3: Positive control (+) diluted 1:1000; Lane 4; Negative control (−). Lanes 5 through 8 show samples from patient A pretreatment (0 weeks, lane 5), after 2 weeks (lane 6), 8 weeks (lane 7), and 14 weeks (lane 8) post AAV-vector injection. Samples from patient B are shown in lanes 9 through 12; pretreatment (0 weeks, lane 9), after 1 week (lane 10), 6 weeks (lane 11), and 8 weeks (lane 12) post AAV-vector injection. (b) Neutralizing antibody titers against AAV before and after treatment with AAV-CMV-hF.IX.

FIGS. 3a and 3b depict F.IX usage and coagulation assays for patients A (top arrow, FIG. 3a) and B (bottom arrow, FIG. 3b). The horizontal line denotes time; the scale at the bottom is marked in 20-day increments. Arrows denote infusion of F.IX concentrate for spontaneous bleeds (thin arrows), or invasive procedures (thick arrows). The thick vertical arrow in the middle of the chart denotes the date of vector infusion. The hatched bar on the timeline denotes the initial 6 week period during which the hF.IX transgene product is expected to be low, based on animal studies. All patients have baseline F.IX levels <1%.

### DETAILED DESCRIPTION OF THE INVENTION

The invention provides methods for delivering rAAV virions comprising a heterologous nucleic acid sequence to humans having preexisting anti-AAV antibodies. As used herein, “preexisting anti-AAV antibodies” is defined as antibodies to one or more of the several wtAAV serotypes (e.g., AAV-1, AAV-2, AAV-3, AAV-4, AAV-5, AAV-6) as well as antibodies to rAAV virions derived from any of the several wtAAV serotypes. The term also encompasses AAV serotypes created from the combination of two or more different AAV serotypes, i.e., a “hybrid” serotype, and includes any rAAV virions derived therefrom. The term further embraces AAV serotypes created using “gene shuffling” techniques and any rAAV virions derived therefrom. By “recombinant AAV virion” or “rAAV virion” is meant an infectious virus composed of an AAV protein shell (i.e., a capsid) encapsulating a heterologous nucleic acid sequence that is flanked by one or more AAV ITRs. The “heterologous nucleic acid sequence” encapsulated includes nucleic acid sequences joined together that are not normally found in association with each other in nature. For example, a heterologous nucleic acid sequence could include a coding sequence flanked by sequences not found in association with the coding sequence in nature. Another example of a heterologous nucleic acid sequence is a coding sequence that is not found in nature (e.g., synthetic sequences having codons different from the native gene). Allelic variation or naturally occurring mutational events do not give rise to heterologous nucleic acid sequences, as used herein. A heterologous nucleic acid sequence can comprise an anti-sense RNA sequence, a ribozyme, or a gene encoding a polypeptide.

In a preferred embodiment, rAAV is produced using a triple transfection system. This system involves the use of three vectors for rAAV virion production, including an AAV helper function vector, an accessory function vector, and an AAV vector. One of skill in the art will appreciate, however, that the nucleic acid sequences encoded by these vectors can be provided on two or more vectors in various combinations. As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors.

The AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type or pseudo-wild-type AAV virions (i.e., AAV virions containing functional rep and cap genes). An example of such a vector, pHLP19 is described in detail in U.S. Pat. No. 6,001,650, the entirety of which is hereby incorporated by reference.

The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of Cap expression products, and AAV capsid assembly. Viral-based accessory functions can be derived from any of the known helper viruses such as adenovirus, herpesvirus (other than herpes simplex virus type-1), and vaccinia virus. In a preferred embodiment, the accessory function plasmid pladeno5 is used (details regarding pladeno5 are described in U.S. Pat. No. 6,004,797, the entirety of which is hereby incorporated by reference). This plasmid provides a complete set of adenovirus accessory functions for AAV vector production, but lacks the components necessary to form replication-competent adenovirus.

The “AAV vector” can be a vector derived from any AAV serotype, including without limitation, AAV-1, AAV-2, AAV-3A, AAV-3B, AAV-4, AAV-5, AAV-6, etc. AAV vectors can have one or more of the wtAAV genes deleted in whole or in part, i.e., the rep and/or cap genes, but retain at least one functional flanking ITR sequences, as necessary for the rescue, replication, and packaging of the AAV virion. Thus, an AAV vector is defined herein to include at least those sequences required in cis for viral replication and packaging (e.g., functional ITRs). The ITRs need not be the wild-type nucleotide sequences, and may be altered, e.g., by the insertion, deletion, or substitution of nucleotides, so long as the sequences provide for functional rescue, replication, and packaging. AAV vectors can be constructed using recombinant techniques that are known in the art to include one or more heterologous nucleic acid sequences flanked with functional AAV ITRs, the incorporation of the heterologous nucleic acid sequence defining a “rAAV vector.”

The invention contemplates the delivery of one or more of several therapeutic nucleotide sequences. In particular, the invention should be construed to include AAV vectors encoding any of the blood coagulation factors, which factors may be delivered, using the methods of the present invention, to the cells of a human having hemophilia and preexisting anti-AAV antibodies for the treatment of hemophilia. Thus, the invention should be construed to include: delivery of Factor IX for the treatment of hemophilia B, delivery of Factor VIII for the treatment of hemophilia A, delivery of Factor X for the treatment of Factor X deficiency, delivery of Factor XI for the treatment of Factor XI deficiency, delivery of Factor XIII for the treatment of Factor XIII deficiency, and delivery of Protein C for the treatment of Protein C deficiency. When referring to any of the blood coagulation factors, it is intended that in addition to the wild-type sequence, biologically active derivatives and/or analogs are encompassed within the scope of these terms. By “biologically active derivatives and/or analogs” is meant molecules derived from the native polypeptide sequence, as well as recombinantly produced or chemically synthesized polypeptides that function in a manner similar to the reference molecule to achieve a desired result. Thus, for example, a biologically active analog of Factor LX, as used herein, encompasses derivatives of the Factor IX polypeptide, including any single or multiple amino acid additions, substitutions, and/or deletions occurring internally or at the amino or carboxy termini thereof, so long as the ability to mediate blood coagulation is maintained.

The invention further includes the delivery of rAAV virions carrying heterologous nucleic acid sequences comprising the DNA of any desired gene that encodes a polypeptide that is defective or missing from a recipient cell, or that encodes a non-native polypeptide having a desired physiological activity, i.e., a therapeutic effect (e.g., an antibacterial function), or a molecule having an anti-sense (e.g., anti-sense mRNA) or ribozyme function. Suitable genes include those used for the treatment of inflammatory diseases, autoimmune, chronic, and infectious diseases including such disorders as AIDS, cancer, neurological diseases, cardiovascular diseases, hypercholesterolemia, various other blood disorders including anemias and thalassemias, genetic defects such as cystic fibrosis, Gaucher disease, adenosine deaminase (ADA) deficiency, emphysema, etc. More specifically, suitable DNA and associated diseases include, but are not limited to: DNA encoding glucose-6-phosphatase, associated with glycogen storage deficiency type 1A; DNA encoding phosphoenolpyruvatecarboxykinase, associated with Pepck deficiency; DNA encoding galactose-1-phosphate uridyl transferase, associated with galactosemia; DNA encoding phenylalanine hydroxylase, associated with phenylketonuria; DNA encoding branched chain α-ketoacid dehydrogenase, associated with Maple syrup urine disease; DNA encoding fumarylacetoacetate hydrolase, associated with tyrosinemia type 1; DNA encoding methylmalonyl-CoA mutase, associated with methylmalonic academia; DNA encoding medium chain acyl CoA dehydrogenase, associated with medium chain acyl CoA dehydrogenase deficiency; DNA encoding ornithine transcarbamylase, associated with ornithine transcarbamylase deficiency; DNA encoding argininsuccinic acid synethase, associated with citrullinemia; DNA encoding low density lipoprotein receptor protein, associated with familial hypercholesterolemia; DNA encoding UDP-glucouronosyltransferase, associated with Crigler-Najjar disease; DNA encoding adenosine deaminase, associated with severe combined immunodeficiency disease; DNA encoding hypoxanthine guanine phosphoribosyl transferase, associated with Gout and Lesch-Nyan syndrome; DNA encoding biotinidase, associated with biotinidase deficiency; DNA encoding β-glucocerebrosidase, associated with Gaucher disease; DNA encoding β-glucuronidase, associated with Sly syndrome; DNA encoding peroxisome membrane protein 70 kDa, associated with Zellweger syndrome; DNA encoding porphobilinogen deaminase, associated with acute intermittent porphyria; DNA encoding α₁ antitrypsin for the treatment of α-1 antitrypsin deficiency (emphysema); DNA encoding erythropoietin for the treatment of anemia due to thalassemia or to renal failure; DNA encoding insulin for the treatment of diabetes; DNA encoding any one of the several cytokines for the treatment of cancer or inflammatory diseases; DNA encoding p52 for the treatment of various cancers; DNA encoding Rb for the treatment of various cancers; and DNA encoding aromatic amino acid decarboxylase for the treatment of Parkinson's disease.

A number of antisense oligonucleotides suitable for use with the present invention in cancer anti-sense therapy or treatment of viral diseases have been described in the art. See, e.g., Han et al., (1991) Proc. Natl. Acad. Sci. USA 88:4313-4317; Uhlmann et al., (1990) Chem. Rev. 90:543-584; Helene et al., (1990) Biochim. Biophys. Acta. 1049:99-125; Agarawal et al., (1988) Proc. Natl. Acad. Sci. USA 85:7079-7083; and Heikkila et al., (1987) Nature 328:445-449. For a discussion of suitable ribozymes, see, e.g., Cech et al., (1992) J Biol. Chem. 267:17479-17482 and U.S. Pat. No. 5,225,347.

Expression of the heterologous nucleic acid sequence is under the control of a promoter/regulatory sequence. By “promoter/regulatory sequence” is meant a DNA sequence that is required for expression. In some instances, the promoter/regulatory sequence may be a core promoter sequence and in other instances, the promoter/regulatory sequence may also include an enhancer sequence and/or other regulatory sequences that are required for expression of the heterologous nucleic acid sequence. The promoter may be one that is constitutive or it may be inducible. If constant expression of the heterologous nucleic acid sequence is desired, then a constitutive promoter is used. Examples of well known constitutive promoters include the immediate-early cytomegalovirus (CMV) promoter, the Rous sarcoma virus promoter, and the like. Numerous other examples of constitutive promoters are well known in the art and can be employed in the practice of the invention.

If controlled expression of the heterologous nucleic acid sequence is desired, then an inducible promoter may be used. In an uninduced state, the inducible promoter is “silent.” By “silent” is meant that little or no heterologous nucleic acid expression is detected in the absence of an inducer; in the presence of an inducer, however, heterologous nucleic acid expression occurs. Often, one can control the level of expression by varying the concentration of inducer. By controlling expression, for example by varying the concentration of an inducer so that an inducible promoter is stimulated more strongly or more weakly, one can affect the concentration of the transcribed product of the heterologous nucleic acid sequence. In the case where the heterologous nucleic acid sequence codes for a gene, one can control the amount of protein that is synthesized. In this manner, it is possible to vary the concentration of therapeutic product. Examples of well known inducible promoters are: an estrogen or androgen promoter, a metallothionein promoter, or an ecdysone-responsive promoter. Numerous other examples are well known in the art and can be employed in the practice of the invention.

In addition to constitutive and inducible promoters (which tend to work in a wide variety of cell or tissue types), there are tissue-specific promoters that can be used to achieve tissue- or cell-specific expression of the heterologous nucleic acid sequence. Well-known examples of tissue-specific promoters include the muscle-specific skeletal α-actin promoter, the muscle-specific creatine kinase promoter/enhancer, and the liver-specific human α1-antitrypsin promoter. There are numerous tissue-specific promoters that are well known in the art and can be employed in the practice of the invention.

Once delivered, the heterologous nucleic acid sequence, contained within the rAAV virion, is expressed to elicit a therapeutic effect. By “therapeutic effect” is meant a level of expression of one or more heterologous nucleic acid sequences sufficient to alter a component of a disease (or disorder) toward a desired outcome or clinical endpoint, such that a patient's disease or disorder shows clinical improvement, often reflected by the amelioration of a clinical sign or symptom relating to the disease or disorder. For example, in the case of Gaucher disease, the rAAV-delivered glucocerebrosidase gene, and its subsequent expression, has been shown to have a therapeutic effect, namely a change in size and/or shape (i.e., a change in morphology) of macrophages, which can lead to an amelioration of a hepatosplenomegalic disorder.

The invention also provides methods for treating hemophilia in patients who have preexisting anti-AAV antibodies. The methods include the delivery of rAAV virions containing a heterologous nucleic acid sequence (i.e., a heterologous gene) encoding a blood coagulation factor, the expression of which results in a therapeutic effect. Delivery methods include intramuscular injection, intravenous or intra-arterial injection, subcutaneous injection, and the like. In one preferred embodiment, the hemophilic patient is injected at least once into muscle tissue with rAAV virions containing a heterologous nucleic acid sequence coding for one of the blood coagulation factors, preferably injection is into one or more slow-twitch fibers of the muscle tissue. The therapeutic effect obtained is a reduction in usage of recombinant Factor IX or Factor IX concentrate and/or a reduction in activated prothromboplastin time (aPTT), aPTT being one measurement of blood clotting, whereby a reduction in aPTT is associated with increased blood clotting ability.

The dose of rAAV virion required to achieve a particular therapeutic effect, e.g., the units of dose in vector genomes/per kilogram of body weight (vg/kg), will vary based on several factors including, but not limited to: the route of rAAV virion administration, the level of heterologous nucleic acid sequence expression required to achieve a therapeutic effect, the specific disease or disorder being treated, a host immune response to the rAAV virion, a host immune response to the heterologous nucleic acid sequence expression product, and the stability of the expression product. One skilled in the art can readily determine a rAAV virion dose range to treat a patient having a particular disease or disorder based on the aforementioned factors, as well as other factors. Using hemophilia as an example, generally speaking, it is believed that, in order to achieve a therapeutic effect, a blood coagulation factor concentration that is greater than 1% of factor concentration found in a normal individual is needed to change a severe disease phenotype to a moderate one. A severe phenotype is characterized by joint damage and life-threatening bleeds. To convert a moderate disease phenotype into a mild one, it is believed that a blood coagulation factor concentration greater than 5% of normal is needed. With respect to treating a hemophilia patient, a dose is provided that is at least 1×10¹⁰ vector genomes per kilogram (vg/kg), preferably between about 1×10¹⁰−1×10¹¹ vg/kg, more preferably between about 1×10¹¹⁻1×10¹² vg/kg, and most preferably between about 1×10¹¹−1×10¹² −1×10¹³ vg/kg to achieve a desired therapeutic effect.

The rAAV virions can be introduced into humans with preexisting anti-AAV antibodies using several techniques. For example, direct intramuscular injection can be used. In one embodiment, rAAV virions are injected into one or more slow-twitch fibers of a muscle. In another embodiment, a catheter introduced into the femoral artery can be used to delivery rAAV virions to the liver via the hepatic artery. Non-surgical means can also be employed, such as the well-known technique endoscopic retrograde cholangiopancreatography (ERCP), to deliver rAAV virions directly to the liver, bypassing the bloodstream altogether. This technique is further described in the Examples below. Other ductal systems, such as the ducts of the submandibular gland, can also be used as portals of entry for delivering rAAV virions into the human with preexisting anti-AAV antibodies.

The invention provides methods for successful rAAV virion transduction leading to therapeutic expression of heterologous nucleic acid sequences in patients with preexisting anti-AAV antibodies. Thus, in the preferred embodiments, recombinant AAV virion delivery, whether by intramuscular injection, ERCP, submandibular gland ductal infusion, hepatic artery, etc., is accomplished without the use of an immune suppressant. For the purposes of clarity and for exemplifying the best mode of the present invention, the discussion that follows exemplifies rAAV virion delivery of human Factor IX to a human with preexisting anti-wtAAV antibodies.

Below are examples of specific embodiments for carrying out the present invention. The examples are offered for illustrative purposes only, and are not intended to limit the scope of the present invention in any way.

### EXAMPLE 1

### Recombinant AAV Virion Production

A rAAV vector, AAV-CMV-hF.IX, is described in U.S. Pat. No. 6,093,392, the entirety of which is hereby incorporated by reference. Briefly, the vector is comprised of a heterologous nucleic acid sequence further comprising the human blood coagulation Factor IX (hF.IX) gene, a 1.4 kb fragment of the first intron of the hF.IX gene, the cytomegalovirus (CMV) immediate early promoter/enhancer, and various other 5′ and 3′ untranslated sequences (e.g., SV40 late polyadenylation sequence) flanked by AAV-2 ITR sequences.

### EXAMPLE 2

### Recombinant AAV Virion Administration

On Day 0, adult hemophilic patients with preexisting anti-AAV antibodies were infused with Factor IX concentrate to bring Factor IX levels up to ˜100% of normal, and, under ultrasound guidance, rAAV virions containing the Factor IX gene were injected percutaneously into 10-12 sites in the vastus lateralis of either or both anterior thighs. Injectate volume at each site was 250-500 μL at a dose of 2×10¹¹ vg/kg, and sites were at least 2 cm apart. Local anesthesia to the skin was provided by ethyl chloride or eutectic mixture of local anesthetics (EMLA). To facilitate subsequent muscle biopsy, the skin overlaying several injection sites was tattooed and the injection coordinates recorded by ultrasound. Patients were then observed in the hospital for 24 h after rAAV virion injection.

### EXAMPLE 3

### Clinical Laboratory Studies

Serum, semen, urine, saliva, and stool samples were collected and subjected to PCR detection of AAV vector sequences. The 5′ primer (5′-AGTCATCGCTATTACCATGG-3′) was derived from the CMV promoter and the 3′ primer (5′-GATTTCAAAGTGGTAAGTCC-3′) was derived from intron I of human Factor IX. Amplified vector sequence yields a PCR fragment of 743 bp. For each sample, a control reaction containing the sample to be tested spiked with vector plasmid (50 copies/μg DNA) was also run to establish that the sample did not inhibit the PCR reaction. For semen, 3 μg of DNA was analyzed (1 μg in each of 3 separate reactions); for saliva, 1 μg; and for urine, serum, and stool, DNA was extracted from 1-2 mL volume and analyzed. The sensitivity of the assay is 50 copies of vector sequence in 1 μg DNA. Serum samples were positive for AAV vector sequences 24 and 48 h pqst-injection and negative at time points thereafter. PCR reactions were performed in a total reaction volume of 100 μL including 1.5 mM MgCl₂ and 0.5 μM of each primer. Following an initial denaturation step (94° C. for 4 min), 35 cycles of the following profile were carried out: Saliva samples were positive 24 h post-injection but negative thereafter. One patient had a positive urine sample 24 h post-injection but was negative thereafter. All other samples were negative for AAV vector sequences, including serum samples taken up to 59 days after AAV vector injection.

### EXAMPLE 4

### Muscle Biopsy Studies

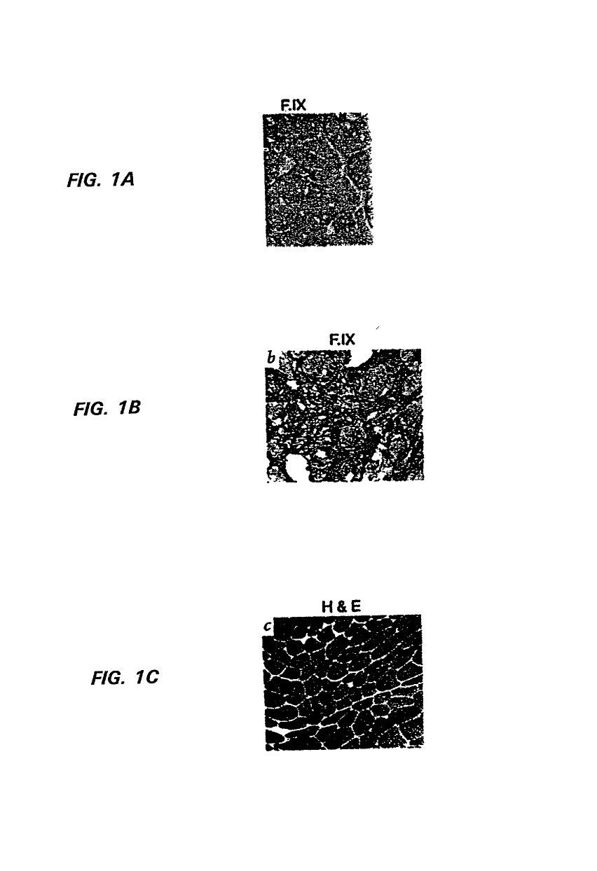

Muscle biopsies were conducted at 2, 6, and 12 months after injection. Studies on skeletal muscle included routine hematoxylin and eosin (H&E) staining using standard techniques that are well known in the art, PCR for vector sequences (1 μg of DNA was analyzed using the methods and primers as discussed in Example 4, supra), Southern blot analysis for detection of vector (using standard techniques well known in the art and described in U.S. Pat. No. 6,093,392, supra), and immunohistochemical staining for Factor IX expression. Biopsied muscle tissue was placed in Optimal Cutting Temperature™ (OCT) (Tissue-TEK OCT 4583 Compound, Sakura Finetek, Torrance, Calif.) in a cryovial, snap-frozen in liquid nitrogen-cooled 2-methyl butane for 7-10 seconds and then immediately transferred to liquid nitrogen and subsequently stored at −80° C. Cryosections were stained using a goat anti-human Factor IX antibody (Affinity Biologicals, Hamilton, Ontario, Canada; 1:800 dilution) as described in U.S. Pat. No. 6,093,392, supra, except that a biotinylated horse anti-goat IgG was used as the secondary antibody (1:200 dilution) for immunoperoxidase staining, which was contained in a kit (Vector Laboratories, Burlingame, Calif.). Sections were counterstained with Myers hematoxylin stain. FIG. 1a shows a lack of Factor IX detected in immunoperoxidase-stained muscle tissue, whereas FIG. 1b shows the presence, by immunoperoxidase staining, of Factor IX from muscle biopsy tissue taken from patients injected with the AAV-CMV-hF.IX vector. Importantly, Factor IX was detected in the extracellular matrix of the muscle tissue, indicating that Factor IX was expressed and secreted from cells comprising the muscle tissue. FIG. 1c depicts normal morphological appearance of muscle tissue from patients injected with the AAV-CMV-hF.IX vector (as visualized using H&E staining) indicating that there had been no inflammatory response or injury due to injected vector, or its transgene product (i.e., Factor IX). PCR and Southern blot analyses detected the presence of the AAV-CMV-hF.IX vector in muscle tissue, thus confirming rAAV virion tranduction.

### EXAMPLE 5

### Antibody Assays

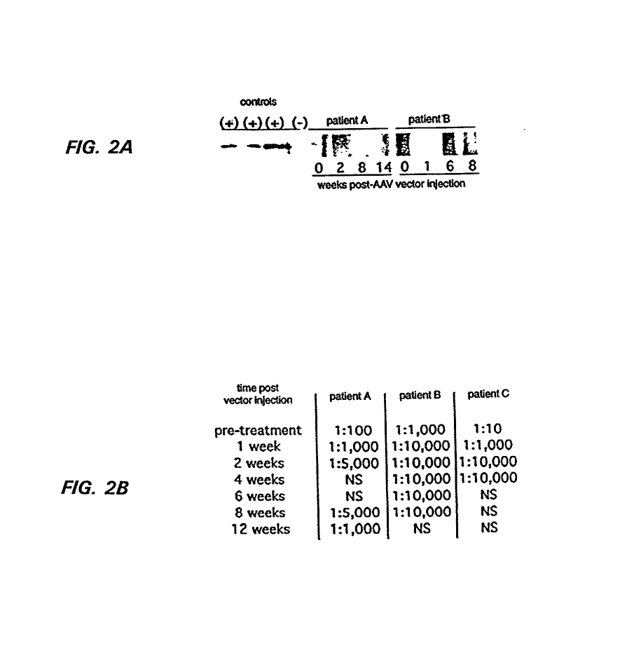

For Patients A, B and C, adeno-associated virus antibody titers were determined by incubating an AAV vector containing the lacZ gene (which encodes for β-galactosidase) with serial dilutions of patient serum, and the resultant cocktail was used to transduce HEK 293 cells. Cells were lysed after 24 h and assayed for enzymatic activity using the o-nitrophenyl β-D-galactopyranoside (ONPG) assay. Samples were read at OD₄₂₀ to measure β-galactosidase activity; sera were scored as positive for neutralizing AAV antibodies if the OD₄₂₀ was ≦50% of that observed when rAAV-lacZ was pre-incubated with negative control mouse sera. Positive samples were titered; AAV neutralizing antibody titers are presented as dilutions that inhibit infection of rAAV-lacZ by 50% based on the ONPG assay. Standard Western blot analysis was then conducted to detect anti-Factor IX antibodies. Purified human Factor IX was electrophoresed on a 7.5% polyacrylamide gel and transferred to a nitrocellulose membrane using an electroblot system (Bio-Rad, Hercules, Calif.). The membrane was incubated with a 1:10000 dilution of the patient's serum sample as primary antibody, and 1:10000 dilution of anti-human IgG peroxidase conjugate using a chemiluminescent substrate (Pierce Chemical Company, Rockford, Ill.) as detecting antibody. FIGS. 2a and 2b depict the results. As shown in FIG. 2a, Western blot analysis for anti-human Factor IX antibodies in serum samples of hemophilia B patients showed a lack of inhibitory and non-inhibitory antibodies to human Factor IX. As shown in FIG. 2b, ONPG assay results showed that all patients had preexisting neutralizing antibody titers to AAV. After AAV-CMV-hF.IX injection, neutralizing antibody titers increased in one patient (patient C) from 1:10 to 1:10,000.

### EXAMPLE 6

### Circulating Factor IX Levels

Circulating plasma Factor IX levels were measured using an automated analyzer (MDA II, Organon-Teknika, Durham, N.C., or MLA Electra 800, GMI, Inc., Clearwater, Minn.). Plasma test samples were mixed with Factor IX-deficient substrate (George King Bio-Medical, Inc., Overland Park, Kans.) and results compared with the degree of correction obtained when dilutions of reference plasma were added to the same Factor IX-deficient substrate. The reference curve was linear down to a lower limit of 0.3%. Table 1 depicts circulating plasma Factor IX levels. Initial circulating plasma Factor IX levels were below 1% prior to AAV-CMV-hF.IX injection. After injection, circulating Factor IX levels exceeded 1.5% in one patient (1% considered sufficient to change the course of hemophilia B from a severe phenotype to a moderate one). Factor IX levels in the other patient reached 0.8% (Table 1).

TABLE 1
Coagulation Data* for Patients A and B

| | Patient A | | | Patient B | | |
|---|---|---|---|---|---|---|
| | F.IX | aPTT | | F.IX | aPTT | |
| Baseline | <1% | | | <0.3% | | |
| Week 6 | <0.3% | 82.9 | | | | |
| Week 8 | 1% | 61 | | <0.3% | 102 | |
| Week 10 | 1.6% | 48 | | 0.3% | 91.2 | |
| Week 12 | 1.4% | 46.8 | | 0.3% | 102.3 | |
| Week 14 | 3.7% | 41 | (post-F.IX infusion) | 3.0% | 52.6 | (post-F.IX infusion) |
| Week 17 | 1.3% | 50.6 | | 0.4% | 72 | |
| Week 18 | 0.8% | 49.4 | | | | |
| Week 20 | 0.5% | 54.1 | | 0.4% | 107 | |
| Week 22 | 0.9% | 53.7 | | | | |
| Week 24 | 0.5% | 52.1 | | 0.8% | 65.5 | |

*Unless otherwise noted, all data points were drawn at least 14 days after most recent Factor IX concentrate infusion.

### EXAMPLE 7

### Coagulation Activity

Activated partial thromboplastin time (aPTT) was measured. Patients'plasma was collected in citrate buffer and clotting times measured by mixing 50 μL of aPTT reagent (Organon-Teknika, Durham, N.C.) with 50 μL of patient plasma. The mixture was incubated at 37° C. for 3 minutes before addition of 50 μL of 25 mM CaCl₂. The clotting time was measured using a fibrometer (FibroSystem; BBL, Cockeysville, Md.). Table 1 depicts aPTT values. As Table 1 shows, a reduction in aPTT was seen in both Patients A and B subsequent to injection with the AAV-CMV-hF.IX vector. The reduction in aPTT was observed for the entire length of the study period, 24 weeks.

### EXAMPLE 8

### Factor IX Concentrate Consumption

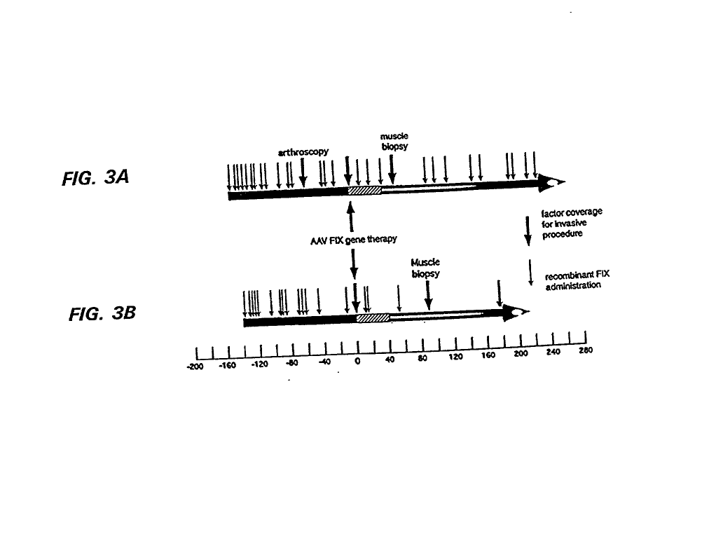

Factor IX concentrate consumption is graphically illustrated in FIGS. 3a and 3b. For Patient A, Factor IX was measured at 1%, with an aPTT of 61 sec, when he presented for muscle biopsy 8 weeks following AAV-CMV-hF.IX injection. On the day of and 1 day following muscle biopsy, Patient A received Factor IX concentrate; 17 days later, with no intervening factor treatment, the Factor IX level was 1.6%, with an aPTT of 48 sec. Ten days later, the Factor IX level was 1.4%, with an aPTT of 47 sec, again with no intervening factor treatment. Ten days later the patient treated himself with concentrate for atypical knee pain, and a Factor IX level drawn 4 days following self-treatment was 3.7%, with an aPTT of 41 sec, reflecting the recent protein infusion. A blood sample drawn 14 days after a subsequent treatment showed a Factor IX level of 1.3%, with an aPTT of 50 sec. Over the ensuing weeks, the Factor IX level was measured in the range of 0.5-1.0%, with aPTT's of about 50 sec. Factor infusion was reduced ˜50% from baseline, and this was sustained over a period of more than 100 days. Patient B's baseline Factor IX level was <0.3%; his baseline factor infusion was ˜2 - 5 times per month. Patient B's Factor IX concentrate consumption decreased by >80%; as for Patient A, this degree of reduction lasted for more than 100 days. FIGS. 3a and 3b depict Factor IX concentrate consumption prior to AAV-CMV-hF.IX injection, and Factor IX concentrate consumption subsequent to vector injection for Patients A and B. Factor IX concentrate consumption prior to vector injection served as a control for analyzing Factor IX concentrate consumption after vector injection.

### EXAMPLE 9

### Predicted Levels of Circulating Factor IX in Humans

On the basis of studies in mice and hemophilic dogs, it was predicted that patients given the dose of 2×10¹¹ vg/kg would not show measurable levels of Factor IX expression (i.e., <0.3%). However, as shown in Table 2, Patient A's Factor IX levels exceeded 1% at the dose of 2×10¹¹ vg/kg. These observations, coupled with the unanticipated reduction in Factor IX concentrate consumption by Patient B suggests that the AAV-CMV-hF.IX vector may be more efficient in humans than in mice or dogs.

TABLE 2
Predicted Levels of Circulating Factor IX in Humans

| | | | Predicted F.IX | Predicted % |
|---|---|---|---|---|
| Dose | F.IX level in | F.IX level in | level in | normal F.IX |
| (vg/kg) | mice* | dogs** | humans*** | level in humans |
| 2 × 10¹¹ | 6 ng/mL | 2-4 ng/mL | 2-6 ng/mL | ≦0.1 |
| 2 × 10¹² | 60 ng/mL | 16 ng/mL | 16-60 ng/mL | 0.3-1.2 |
| 1 × 10¹³ | 300 ng/mL | 80 ng/mL | 80-300 ng/mL | 1.8-6 |

*Predicted plasma Factor IX level in mice based on mouse experimental data.
**Predicted plasma Factor IX level in dogs based on canine experimental data.
***Extrapolated from studies in animals.

### EXAMPLE 10

### Recombinant Adeno-Associated Virus Virion Delivery via Hepatic Artery to Humans with Preexisting anti-AAV Antibodies

Using the standard Seldinger technique, the common femoral artery is cannulated with an angiographic introducer sheath. The patient is then heparinized by IV injection of 100 U/kg of heparin. A pigtail catheter is then advanced into the aorta and an abdominal aortogram is performed. Following delineation of the celiac and hepatic arterial anatomy, the proper hepatic artery (HA) is selected using a standard selective angiographic catheter (Simmons, Sos-Omni, Cobra or similar catheters). Selective arteriogram is then performed using a non-ionic contrast material (Omnipaque or Visipaque). The catheter is removed over a 0.035 wire (Bentsen, angled Glide, or similar wire). A 6F Guide-sheath (or guide-catheter) is then advanced over the wire into the common HA. The wire is then exchanged for a 0.018 wire (FlexT, Microvena Nitenol, or similar wire) and a 6×2 Savvy balloon is advanced over the wire into the proper HA distal to the gastroduodenal artery. The wire is then removed, the catheter tip position confirmed by hand injection of contrast into the balloon catheter, and the lumen flushed with 15 mL of heparinized normal saline (NS) to fully clear the contrast. Prior to infusion of the vector, the balloon is inflated to 2 atm to occlude the flow lumen of the HA. The vector is then infused over 7-15 minutes. The balloon lumen is then flushed with 2 cc of heparinized NS over a period of 5-7 minutes. The balloon is deflated and catheters removed. A diagnostic arteriogram of the femoral puncture site is then performed in the ipsilateral anterior oblique projection and the puncture site closed using either a 6 F Closer (Perclose, Inc. Menlo Park, Calif.) or a 6 F Angioseal.

### EXAMPLE 11

### In Vivo Gene Transfer of DNA Encoding Human Factor IX by Injecting Recombinant Adeno-Associated Virus Virions Directly into the Liver

Recombinant AAV virions containing the AAV-CMV-hF.IX vector is delivered directly to the liver of a human with preexisting anti-AAV antibodies by means of endoscopic retrograde cholangiopancreatography (ERCP). For the procedure, the patient will lie on his left side on an examining table in an x-ray room. The patient will be given medication to help numb the back of his throat and a sedative to help him relax during the exam. The patient will swallow the endoscope, and the gastroenterologist will then guide the endoscope through the esophagus, stomach, and duodenum until it reaches the spot where the ducts of the biliary tree and pancreas open into the duodenum. At this time, the patient will lie flat on his stomach, and the gastroenterologist will pass a catheter through the endoscope. Through the catheter, the gastroenterologist will inject a dye into the ducts and x-rays will be taken to ensure that there are no blockages. Once the X-ray examination is complete and no complications are evident, an injectate of rAAV virions containing the AAV-CMV-hF.IX vector is then delivered via the catheter. Once the rAAV virion injection is complete, the endoscope is removed and the patient is monitored for ˜60 min.

Many modifications and variations of this invention, as will be apparent to one of ordinary skill in the art can be made to adapt to a particular situation, material, composition of matter, process, process step or steps, to preserve the objective, spirit and scope of the invention. All such modifications are intended to be within the scope of the claims appended hereto without departing from the spirit and scope of the invention. The specific embodiments described herein are offered by way of example only. The invention is not to be limited by the specific embodiments that have been presented herein by way of example.

We claim:

1. A method of administering recombinant adeno-associated virus (rAAV) virions to a human, comprising:
(a) providing at least one rAAV virion, said at least one rAAV virion comprising a vector further comprising a heterologous nucleic acid sequence; and
(b) delivering said rAAV virions to a human wherein said human has preexisting anti-AAV antibodies;
(c) wherein said heterologous nucleic acid sequence is expressed.

2. The method of claim 1, wherein said preexisting anti-AAV antibodies are anti-AAV-2 antibodies.

3. The method of claim 1, wherein expression of said heterologous nucleic acid sequence results in a therapeutic effect.

4. The method of claim 1, wherein said heterologous nucleic acid sequence codes for a polypeptide.

5. The method of claim 4, wherein said polypeptide is Factor IX.

6. The method of claim 5, wherein said Factor IX is secreted into an extracellular space.

7. The method of claim 5, wherein said Factor IX is secreted into a blood vessel.

8. The method of claim 1, wherein the delivering of said rAAV virions to said human is by injection to a muscle.

9. The method of claim 8, wherein said injection is to one or more slow-twitch muscle fibers of said muscle.

10. The method of claim 8, wherein said injection is performed at least once on said muscle.

11. The method of claim 1, wherein the delivering of said rAAV virions to said human is by injecting into a duct of a secretory gland.

12. The method of claim 11, wherein the secretory gland is a liver.

13. A method of treating hemophilia in a human, comprising:
(a) providing at least one recombinant adeno-associated virus (rAAV) virion, said rAAV virion comprising a vector further comprising a heterologous nucleic acid sequence further comprising a gene encoding a blood coagulation factor; and
(b) delivering said rAAV virions to said human wherein said human has preexisting anti-AAV antibodies; and
(c) wherein said blood coagulation factor is expressed at a level having a therapeutic effect.

14. The method of claim 13, wherein said preexisting anti-AAV antibodies are anti-AAV-2 antibodies.

15. The method of claim 13, wherein said blood coagulation factor is Factor IX.

16. The method of claim 15, wherein said Factor IX is human Factor IX.

17. The method of claim 15, wherein said Factor IX is secreted into an extracellular space.

18. The method of claim 15, wherein said Factor IX is secreted into a blood vessel.

19. The method of claim 17, wherein said Factor IX is human Factor IX.

20. The method of claim 18, wherein said Factor IX is human Factor IX.

21. The method of claim 13, wherein the delivering of said rAAV virion to said human is by injection to a muscle.

22. The method of claim 19, wherein said injection is to one or more slow-twitch muscle fibers.

23. The method of claim 20, wherein said injection is to one or more slow-twitch muscle fibers.

24. The method of claim 21, wherein said injection is performed at least once on said muscle.

25. The method of claim 22, wherein said injection is performed at least once on said muscle.

26. The method of claim 23, wherein said injection is performed at least once on said muscle.
